(19) United States
(12) Patent Application Publication
(10) Pub. No.: US 2021/0315741 A1
(43) Pub. Date: Oct. 14, 2021

(54) COMPRESSION STOCKING FOR HEMIPLEGIA PATIENT

(71) Applicant: AJOU UNIVERSITY INDUSTRY-ACADEMIC COOPERATION FOUNDATION, Suwon-si (KR)

(72) Inventor: Ji Man HONG, Yongin-si (KR)

(73) Assignee: AJOU UNIVERSITY INDUSTRY-ACADEMIC COOPERATION FOUNDATION, Suwon-si (KR)

(21) Appl. No.: 17/416,013

(22) PCT Filed: Dec. 18, 2019
(86) PCT No.: PCT/KR2019/017982

§ 371 Date: Jun. 18, 2021

(30) Foreign Application Priority Data
Dec. 21, 2018 (KR) 10-2018-0166985
Dec. 6, 2019 (KR) 10-2019-0161486

(51) Int. Cl. A61F 13/08

(52) U.S. Cl. CPC A61F 13/08

(57) ABSTRACT

A According to the present disclosure, there is provided a compression stocking for a hemiplegia patient worn on the hemiplegia patient's arm or leg, including: a main body having an observation portion configured to observe a corresponding body portion so as to check a vascular lesion of the patient, in which the main body has different compression degrees depending on portions of the arm or the leg. According to the present disclosure, it is possible to correct and treat the acute unilateral limb problem by wearing the compression stocking on the arm or leg requiring treatment of the hemiplegia patient, prevent thrombus by compressing the arm or leg with different pressures in a state where the compression stocking for a hemiplegia patient is worn on the hemiplegia patient's arm or leg, and check the vascular lesion of the patient through the observation portion formed at the end thereof in a state where the compression stocking for a hemiplegia patient is worn on the hemiplegia patient's arm or leg.

### TECHNICAL FIELD

The present disclosure relates to a compression stocking, and more particularly, to a compression stocking for a hemiplegia patient for effectively correcting and treating an acute hemiplegic problem that may occur in a hemiplegia patient.

### BACKGROUND

In general, the orthodoxy of the human brain is that respective portions of the brain have different functions. For this reason, in a case where a specific portion of the brain is damaged by a stroke, traumatic brain injury, a brain tumor, or the like, hemiplegia may occur.

The hemiplegia refers to a condition in which a movement disorder occurs on one side of the upper and lower limbs or the face. The hemiplegia patients suffer from acute unilateral limb problems (hemiplegia and edema of the arm and leg, venous thrombus, and non-responsiveness to unilateral sensation) that can be caused by motor-sensory dysfunction in the opposite arm and leg to the brain mainly damaged by brain injuries (various unilateral brain injuries such as stroke, brain trauma, and congenital encephalopathy).

In the hemiplegia caused by various brain injuries, it is known that moving the immobile hemiplegic limb quickly is very important for quick and effective recovery of the paralysis.

Because the hemiplegic limb cannot move on by itself, the hemiplegic limb is vulnerable to a secondary injury, and skin damage or pressure ulcers due to repeated physical stimulation or gravity on the limbs can easily occur.

In particular, the lower limb portion is known to cause venous thrombosis because a flow rate of the vein is slow and retention is easy to occur, and thereby compression is required to prevent this.

As the prior art for of the present disclosure, Korean Patent Unexamined Publication No. 2014-0029172 may be exemplified.

### SUMMARY OF INVENTION

### Technical Problem

The present disclosure has been devised to solve the above problems, and an object of the present disclosure is to provide a compression stocking for a hemiplegia patient, which can be worn on an arm or leg required for treatment of a hemiplegia patient to correct and treat acute unilateral limb problem.

Another object of the present disclosure is to provide a compression stocking for a hemiplegia patient, which can prevent thrombus by compressing the arm or leg with a pressure different those of other portions in a state where the compression stocking is worn on the arm or leg of the hemiplegia patient.

Another object of the present disclosure is to provide a compression stocking for a hemiplegia patient, which allows the vascular lesion to be checked through an observation portion formed at an end of the arm or leg of the hemiplegia patient in a state where the compression stocking is worn on the arm or leg of the hemiplegia patient.

Another object of the present disclosure is to provide a compression stocking for a hemiplegia patient, which can prevent the compression stocking from slipping down in a state where the compression stocking is worn on the arm or leg of the hemiplegia patient.

In addition, another object of the present disclosure is to provide a compression stocking for a hemiplegia patient, which can prevent abduction of the patient's shoulder by additionally connecting an abduction prevention portion which surrounds the hemiplegia patient's shoulder portion.

### Solution to Problem

In order to achieve the objects described above, according to the present disclosure, there is provided a compression stocking for a hemiplegia patient worn on the hemiplegia patient's arm or leg, including: a main body having an observation portion configured to observe a corresponding body portion so as to check a vascular lesion of the patient, in which the main body has different compression degrees depending on portions of the arm or the leg.

The compression stocking for a hemiplegia patient may further include a shock absorbing portion disposed at a position corresponding to the patient's joint portion to absorb a shock applied to the joint.

The observation portion may be formed of at least one observation hole therethrough to expose a corresponding portion of the patient's body. Here, the observation hall may be formed to correspond to the finger end of the upper limb or the toe end of the lower limb.

As alternative, the observation portion may include a transparent screen made of a transparent material so as to expose a corresponding portion of the patient's body.

As another alternative, the observation portion may be a cover that is provided to cover the finger end of the upper limb or the toe end of the lower limb and is detachably coupled to the main body.

It is preferable that a compression degree of the main body increase as moving away from the patient's body. Here, a compression degree of a portion corresponding to the patient's calf may be 70% of a compression degree of a portion corresponding to the patient's ankle. In addition, a compression degree of a portion corresponding to the patient's thigh may be 50% of a compression degree of a portion corresponding to the patient's ankle.

The compression stocking for a hemiplegia patient may further include a loosening prevention portion that is disposed at one end of the main body and prevents the main body from being loosened by applying a certain pressure to the user's body.

The loosening prevention portion may include an elastic band that is disposed along an inner circumference of one end of the main body.

The compression stocking for a hemiplegia patient may further include a slipping prevention portion that is disposed at a portion corresponding to the patient's lower limb sole to prevent the patient from slipping.

The compression stocking for a hemiplegia patient may further include an abduction prevention portion that is disposed at a portion corresponding to the patient's upper limb joint to prevent the patient's shoulder from turning.

### Advantageous Effects

According to the present disclosure as described above, the compression stocking for a hemiplegia patient can be worn on the hemiplegia patient's arm or leg requiring the treatment to correct and treat the acute unilateral problem.

In addition, according to the present disclosure, thrombus can be prevented by applying compression with different pressures for each portion in a state where the compression stocking for a hemiplegia patient is worn on the hemiplegia patient's arm or leg.

Also, according to the present disclosure, the vascular lesion of the patient can be checked through the observation portion formed at the patient's arm end or leg end in a state where the compression stocking for a hemiplegia patient is worn on the hemiplegia patient's arm or leg.

In addition, according to the present disclosure, the stocking can be prevented from being loosened in a state where the compression stocking for a hemiplegia patient is worn on the hemiplegia patient's arm or leg.

In addition, according to the present disclosure, the abduction prevention portion surrounding the hemiplegia patient's shoulder portion can be additionally connected to prevent abduction of the patient's shoulder.

### BRIEF DESCRIPTION OF THE DRAWINGS

FIG. 1 illustrates a compression stocking for a hemiplegia patient according to an embodiment of the present disclosure, and is a using state view illustrating a state of being worn on the lower limb.

FIG. 2 illustrates a compression stocking for a hemiplegia patient according to an embodiment of the present disclosure, and is a using state view illustrating a state of being worn on the upper limb.

FIG. 3 is a bottom view illustrating an example of a configuration of a slipping prevention portion used in the present disclosure.

FIG. 4 is a view illustrating a compression stocking for a hemiplegia patient according to another embodiment of the present disclosure.

### BEST MODE FOR INVENTION

Hereinafter, preferred embodiments according to the present disclosure will be described in detail with reference to the accompanying drawings.

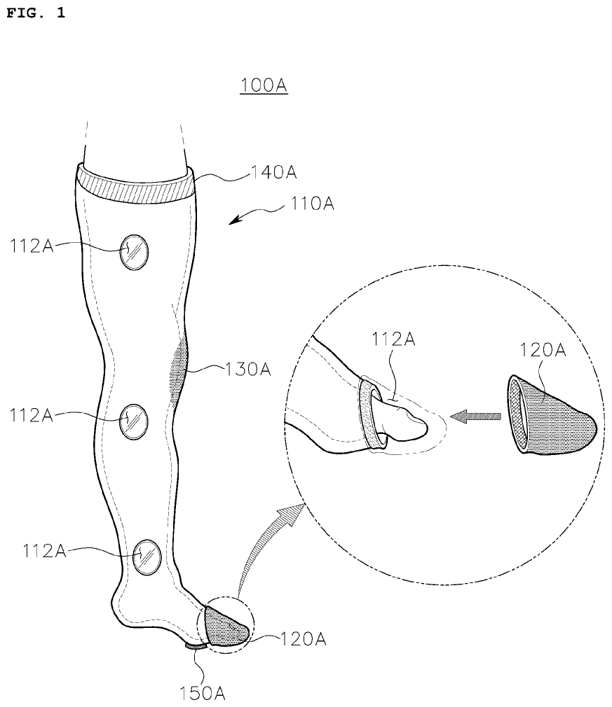

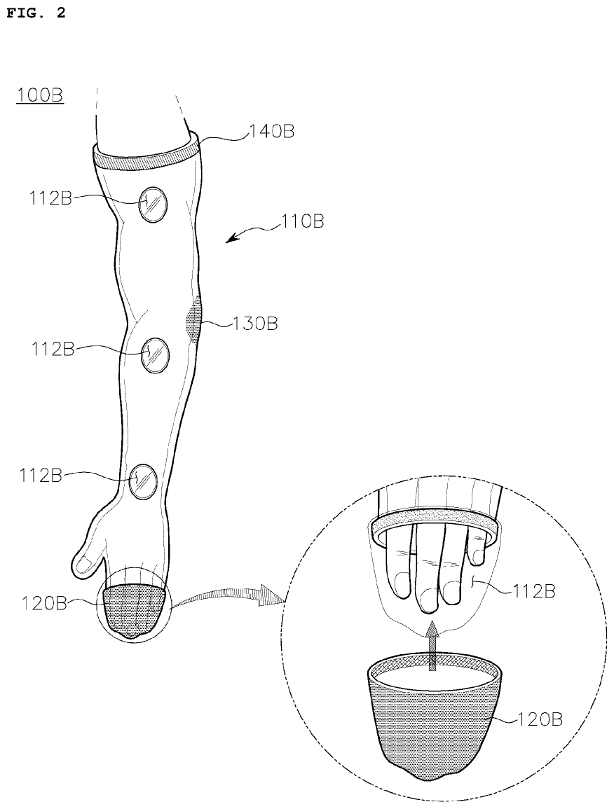

FIGS. 1 and 2 illustrate a compression stocking for a hemiplegia patient according to an embodiment of the present disclosure, in which FIG. 1 is a using state view illustrating a state where the compression stocking for a hemiplegia patient is worn on the lower limb (leg) and FIG. 2 is a using state view illustrating a state where the compression stocking for a hemiplegia patient is worn on the upper limb (arm).

Referring to the drawings, compression stockings 100A and 100B according to the embodiment of the present disclosure include main bodies 110A and 110B.

The main bodies 110A and 110B are provided in a form that can be worn on the leg or arm of a hemiplegia patient. The main body 110A may be made of a fabric having a predetermined elasticity, but the material is not limited thereto.

The main bodies 110A and 110B can press with a predetermined pressure the worn leg or arm. In this case, the main bodies 110A and 110B can apply the pressure with different compression degrees depending on portions of the leg or arm.

Hereinafter, the compression degree with which the main body 110A of the compression stocking 100A worn on the patient's leg compresses the leg will be described.

The more the portion away from the patient's body, the greater the main body 110A can compress. That is, when the compression degree of the patient's ankle is set to 100%, the patient's calf can be compressed with a compression degree of 70%. In addition, the patient's thigh can be compressed with a compression degree of 50%.

On the other hands, the compression degree of the main body 110B in the compression stocking 100B worn on the hemiplegia patient's arm can be set as follows. When the patient's arm has the compression degree of 100% on the wrist, a portion between the wrist and the elbow can be compressed with the compression degree of 70%, and a portion between the elbow and the shoulder can be compressed with the compression degree of 50%.

Here, the main bodies 110A and 110B may have different compression degrees by adjusting weaving intervals during weaving of the fabric, or by weaving with yarns having different elasticity depending on portions.

On the other hands, the main bodies 110A and 110B are provided with observation portions 112A and 112B for observing a vascular lesion that may occur at an end of the patient's arm or leg. The observation portions 112A and 112B allow vascular lesion generating portions to be exposed, so that the vascular lesion generating portions can be observed from the outside.

Here, the observation portions 112A and 112B may be observation holes formed through the body portions 110A and 110B to expose patient's body portions. One or more of these observation holes 112A and 112B may be formed on the main bodies 110A and 110B.

The observation holes 112A and 112B may be each formed in any portion of the patient's leg or arm as illustrated in the drawings.

As another embodiment, the observation holes 112A and 112B are formed at portions of the main bodies 110A and 110B corresponding to the patient's toes or fingers so that the vascular lesion which may be generated in the patient's toes or fingers may also be checked therethrough.

In the drawing, although four toes or fingers are simultaneously exposed through the observation holes 112A and 112B, the toe or finger may be individually exposed by forming the observation hole 112A or 112B to correspond to the toe or finger.

Otherwise, by attaching transparent screens made of a predetermined material such as vinyl or acrylic to the observation holes 112A and 112B, it is possible to prevent a problem such as contamination of the patient's skin by invasion of foreign substances while performing observation through the observation holes 112A and 112B.

As another embodiment, the observation holes 112A and 112B formed at the portions corresponding to the toes or fingers may be provided with covers 120A and 120B having a shape that can cover the end portion of the toes or fingers. In this case, it is preferable that the covers 120A and 120B are detachably coupled to the main bodies 110A and 110B. To this end, the attachment and detachment of the covers 120A and 120B may be performed by attachment and detachment means such as Velcro.

Here, in a case where it is necessary to observe the vascular lesion on the patient's leg or arm, the observation can be performed by detaching the covers 120A and 120B from the main bodies 110A and 110B, and after the observation is completed, the covers 120A and 120B are coupled to the main bodies 110A and 110B to protect the patient's skin. In this case, if the covers 120A and 120B are made of a transparent material, the covers 120A and 120B may serve as the transparent screens disposed in the observation holes 112A and 112B.

On the other hands, the compression stockings 100A and 100B of the present disclosure as described above may further include shock absorbing portions 130A and 130B disposed at positions corresponding to the patient's joint portion. These shock absorbing portions 130A and 130B serve to absorb the shock applied to the patient's joints, and may be made of a conventional sponge or the like, but the material is not limited thereto.

In addition, the compression stockings 100A and 100B according to the present disclosure may further include loosening prevention portions 140A and 140B.

In the following description, each one end of the main bodies 110A and 110B refers to a portion corresponding to the pelvic joint or the shoulder joint in the patient's leg or arm, and the other end of the main bodies 110A and 110B refers to a portion corresponding to the patient's foot or hand.

The loosening prevention portions 140A and 140B are disposed inside one end of the main bodies 110A and 110B worn on the patient's leg or arm to prevent the main bodies 110A and 110B worn on the patient's leg or arm from being loosened.

The loosening prevention portions 140A and 140B include an elastic band that compresses a portion corresponding to the pelvic joint or the shoulder joint in the patient's leg or arm with a predetermined pressure.

In addition, the compression stocking 100A according to the present disclosure may further include a slipping prevention portion 150A.

The slipping prevention portion 150A is disposed at the lower portion of the other end of the main body 110A corresponding to the sole of the patient's foot, and prevents the patient from slipping and falling while walking.

Here, the slipping prevention portion 150A may include a predetermined elastic body such as rubber.

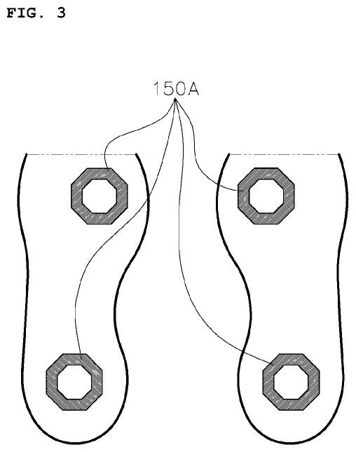

FIG. 3 is a bottom view illustrating an example of a configuration of the slipping prevention portion using in the present disclosure.

Referring to FIG. 3, the slipping prevention portion 150A may be formed in the form of a predetermined octagonal ring in the heel portion of the main body 110A on the outside of the portion corresponding to the patient's sole. In the drawing, the slipping prevention portions 150A are disposed in the heel portions, respectively, but the disposition position may be changed according to the user's need, such as being disposed on the entire sole of the patient's foot.

The slipping prevention portion 150A is not limited to the form illustrated in the drawing, as long as it can prevent the patient from slipping while walking, and may be formed in various forms.

Also, the compression stocking 100B according to the present disclosure may further include an abduction prevention portion 160B.

The abduction prevention portion 160B extends from one end of the main body 110B to the patient's shoulder to surround the patient's shoulder. The abduction prevention portion 160B prevents shoulder abduction with which the patient's shoulder is lifted.

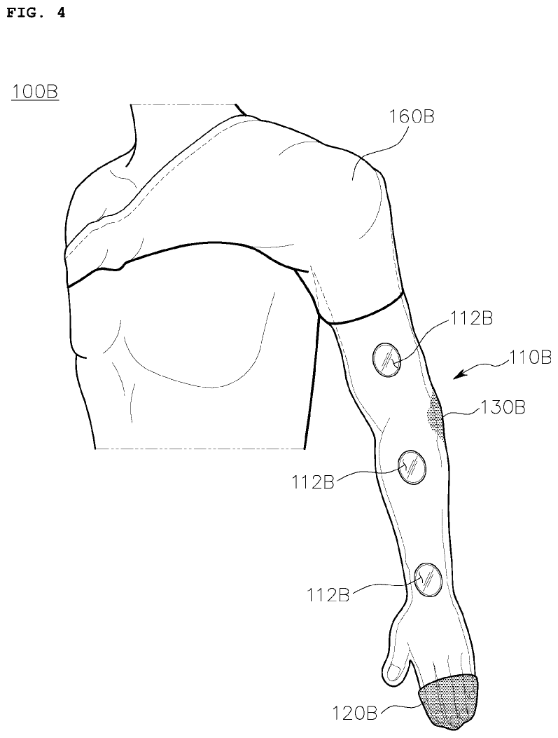

FIG. 4 illustrates a compression stocking for a hemiplegia patient according to another embodiment of the present disclosure and illustrates a state where the abduction prevention portion is connected.

An abduction prevention portion 160B is provided in a form of wrapping the patient's shoulder portion. Here, one side of the abduction prevention portion 160B wraps a side of the patient's body in the patient's chest side and then extends so as to be connected to the other side of the abduction prevention portion 160B through the patient's back.

As described above, according to the present disclosure, it is possible to correct and treat the acute unilateral limb problem by wearing the compression stocking on the arm or leg requiring treatment of the hemiplegia patient, prevent thrombus by compressing the arm or leg with different pressures in a state where the compression stocking for a hemiplegia patient is worn on the hemiplegia patient's arm or leg, and check the vascular lesion of the patient through the observation portion formed at the end thereof in a state where the compression stocking for a hemiplegia patient is worn on the hemiplegia patient's arm or leg.

Although the present disclosure has been described with reference to the embodiments illustrated in the drawings, it will be understood by those skilled in the art that the embodiments are merely exemplary and various modifications and equivalent other embodiments are possible therefrom. Accordingly, the true technical protection scope of the present disclosure should be determined by the technical idea of the appended claims.

1. A compression stocking for a hemiplegia patient worn on the hemiplegia patient's arm or leg, comprising:
a main body having an observation portion configured to observe a corresponding body portion so as to check a vascular lesion of the patient,
wherein the main body has different compression degrees depending on portions of the arm or the leg.

2. The compression stocking for a hemiplegia patient according to claim 1, further comprising:
a shock absorbing portion disposed at a position corresponding to the patient's joint portion to absorb a shock applied to the joint.

3. The compression stocking for a hemiplegia patient according to claim 1,
wherein the observation portion is formed of at least one observation hole therethrough to expose a corresponding portion of the patient's body.

4. The compression stocking for a hemiplegia patient according to claim 3,
wherein the observation hall is formed to correspond to the finger end of the upper limb or the toe end of the lower limb.

5. The compression stocking for a hemiplegia patient according to claim 1,
wherein the observation portion includes a transparent screen made of a transparent material so as to expose a corresponding portion of the patient's body.

6. The compression stocking for a hemiplegia patient according to claim 1,
wherein the observation portion is a cover that is provided to cover the finger end of the upper limb or the toe end of the lower limb and is detachably coupled to the main body.

7. The compression stocking for a hemiplegia patient according to claim 1,
wherein a compression degree of the main body increases as moving away from the patient's body.

8. The compression stocking for a hemiplegia patient according to claim 1,
wherein a compression degree of a portion corresponding to the patient's calf is 70% of a compression degree of a portion corresponding to the patient's ankle.

9. The compression stocking for a hemiplegia patient according to claim 1,
wherein a compression degree of a portion corresponding to the patient's thigh is 50% of a compression degree of a portion corresponding to the patient's ankle.

10. The compression stocking for a hemiplegia patient according to claim 1, further comprising:
a loosening prevention portion that is disposed at one end of the main body and prevents the main body from being loosened by applying a certain pressure to the user's body.

11. The compression stocking for a hemiplegia patient according to claim 10,
wherein the loosening prevention portion includes an elastic band that is disposed along an inner circumference of one end of the main body.

12. The compression stocking for a hemiplegia patient according to claim 1, further comprising:
a slipping prevention portion that is disposed at a portion corresponding to the patient's lower limb sole to prevent the patient from slipping.

13. The compression stocking for a hemiplegia patient according to claim 1, further comprising:
an abduction prevention portion that is disposed at a portion corresponding to the patient's upper limb joint to prevent the patient's shoulder from turning.
